(19) United States
(12) Patent Application Publication
(10) Pub. No.: US 2010/0317584 A1
(43) Pub. Date: Dec. 16, 2010

(54) PHARMACEUTICAL COMPOSITION, PACKAGE AND METHOD FOR RAPIDLY REDUCING THE URIC ACID IN BLOOD

(75) Inventors: William T. H. CHANG, Taipei (TW); Hsi Ying Chen, Taipei (TW); Yi Chieh Wang, Taipei (TW)

Correspondence Address: LADAS & PARRY LLP, 26 WEST 61ST STREET, NEW YORK, NY, 10023, US

(73) Assignee: LYTONE ENTERPRISE, INC.

(21) Appl. No.: 12/861,049

(22) Filed: Aug. 23, 2010

Related U.S. Application Data
(63) Continuation of application No. 11/975,969, filed on Oct. 23, 2007, now abandoned.

(30) Foreign Application Priority Data
Oct. 22, 2007 (EP) 07020633.9

(51) Int. Cl. A61K 38/05; A61P 7/00; A61P 9/10; A61P 19/02; A61P 29/00

(52) U.S. Cl. 514/13.5

(57) ABSTRACT

The present invention relates to a pharmaceutical composition for rapidly reducing the uric acid level in blood in a subject, which comprises anserine in an amount of greater than 50 mg to about 600 mg. The present invention further relates to a package comprising the pharmaceutical composition comprising anserine, and a label and/or an insert indicating that the composition is administered to a subject to provide anserine in an amount of greater than 50 mg to about 600 mg per dose. In addition, the present invention relates to a method for rapidly reducing the uric acid level in blood in a subject, which comprises administering anserine in an amount of greater than 50 mg to about 600 mg to the subject.

### FIELD OF THE INVENTION

The present invention relates to the administration of anserine in an amount greater than 50 mg to about 600 mg so as to rapidly reduce the uric acid level in blood in a subject.

### BACKGROUND OF THE INVENTION

Uric acid is the final product of purine metabolism in human beings. The uric acid level in blood is a function of the balance between the breakdown of purines and the rate of uric acid excretion. For years, hyperuricemia has been associated with or thought to be the same as glut, but uric acid has now been identified as a marker for a number of metabolic and hemodynamic abnormalities. A high uric acid level in blood induces gout, and is normally related to symptoms such as muscle spasm, localized swelling, inflammation, joint pains, muscle fatigue, feelings of stress, and myocardial infraction.

Many commercialized drugs have been used to treat gout, for example, Benzbromarone (URINORM), Probenecid, Allopurinol, Bucolome, Cinchophan and Colchicine. These drugs work by, for example, inhibiting the formation of uric acid, removing excessive uric acid, acting on the kidneys so as to help to eliminate uric acid, inhibiting the activity of xanthine oxidase for converting xanthine into uric acid, and accelerating the excretion of uric acid. However, these drugs are known to have hepatotoxicity, and cause a number of side effects, such as urinary calculus, gastrointestinal obstruction, jaundice, and anemia. These drugs are not reported to be capable of rapidly reducing the uric acid level in blood in a subject.

It is known that some dipeptides are useful in lowering the uric acid level in blood. For example, ROC (Taiwan) Patent No. I280136 (ROC (Taiwan) Patent Application No. 092114798) discloses a composition for reducing the uric acid in a subject, which comprises an effective amount of one or more dipeptides consisting of histidine or functional equivalent thereof and alanine or functional equivalent thereof. Exemplary dipeptides mentioned in the ROC patent include, carnosine, anserine, carcinine, and ophidine. It only teaches that the composition is administered orally to provide an amount of about 8 to about 50 mg, preferably about 10 mg to about 45 mg, of the dipeptide(s) per day, but does not disclose or suggest any route that may rapidly reduce the uric acid level in blood in a subject by administering dipeptide(s).

Anserine (β-alanyl-1-methyl-L-histidine) is a highly stable dipeptide, which has a pK value of approximately 7.1, and remains intact under low pH (<3.0). Anserine naturally exists in the skeletal muscle tissue and brain tissue of vertebrates, and is formed by a peptide bond between β-alanine and 1-methyl-L-histidine (see Tolkachevskaya, NF. 1929. Z Physiol Chem. 185:28-32.). In 1938, it was reported that anserine is an ideal physiological acid-base buffering agent. Due to its physiological acid-base buffering capability, anserine is effective in eliminating the lipid peroxidation promoted by changing the pH values of biological systems. For a subject with acidemia who suffers from gout, the administration of anserine acting as an acid-base buffering agent helps to adjust the pH value of blood, and is advantageous in lowering the uric acid level in blood. It should be understood that 40% of the buffering capability of fast-twitch muscle fibers is contributed by dipeptides in a mobile phase, while the remaining proportion of the buffering capability of fast-twitch muscle fibers is contributed by muscle proteins which are essentially in a fixed phase (see Bate-Smith, E C. 1938, “The buffering of muscle in rigour: protein, phosphate and carnosine.,” J. Physiol. 92:336-43.). This shows that in highly structured muscle cells, local acid-base gradient is composed of buffering assistance of the mobile phase and buffering suppression of the fixed phase.

Moreover, anserine has various antioxidant properties, and is useful as a free radical scavenger or a metal chelating agent. Subjects suffering from inflammation for a long period of time normally have a higher in vivo free radical concentration. The administration of anserine acting as a free radical scavenger is advantageous to eliminate free radicals.

Anserine is also effective in inhibiting the lipid peroxidation promoted by iron, heamoglobin, lipid peroxidase, and singlet oxygen (see Babizhayev M A et. al., 1994, “L-carnosine (beta-alanyl-L-histidine) and carcinine (beta-alanylhistamine) act as natural antioxidants with hydroxyl-radical-scavenging and lipid-peroxidase activities,” Biochem J. 304 (Pt 2): 509-516, and Kohen R. et. al., 1988, “Antioxidant activity of carnosine, homocarnosine, and anserine present in muscle and brain,” Proc Natl Acad Sci USA. 85(9):3175-3179.).

In addition, anserine can ameliorate in vivo oxidative stress caused by fatigue, and enhance oxygen radical absorbance capacity in organisms. Anserine may also act as a precursor of transmitters in central histaminergic nervous system. Through the metabolism of histidine contained in anserine, anserine can effectively adjust the concentration of histamine in central nervous system, lower the concentration of cortisol in blood, and cause a relief of fatigue (see Aoi W. et. al., 2006, “Exercise and functional foods.,” Nutr J. 5: 15-22, and U.S. Pat. No. 6,680,294.

However, commercially available drugs for treating gout comprising anserine are not reported to have an efficacy of rapidly reducing the uric acid level in blood in a subject.

There is still a need to develop a new drug which can rapidly reduce the uric acid level in blood in a subject, thereby treating gout and alleviating the symptoms associated with a high uric acid level in blood.

### SUMMARY OF THE INVENTION

The inventors have surprisingly discovered that the administration of anserine in an amount of greater than 50 mg to about 600 mg per dose can rapidly reduce the uric acid level in blood in the subject.

Therefore, the present invention relates to a pharmaceutical composition for rapidly reducing the uric acid level in blood in a subject, which comprises anserine in an amount of greater than 50 mg to about 600 mg.

The present invention further relates to a package comprising the pharmaceutical composition of the invention, and a label and/or a packing insert indicating that the composition is administered to a subject to provide anserine in an amount of greater than 50 mg to about 600 mg per dose.

Moreover, the present invention relates to a method for rapidly reducing the uric acid level in blood in a subject, which comprises administering anserine in an amount of greater than 50 mg to about 600 mg to the subject.

In addition, the present invention relates to the use of anserine in the manufacture of a medicament for rapidly reducing the uric acid level in blood in a subject, wherein the medicament comprises anserine in an amount of greater than 50 mg to about 600 mg.

### BRIEF DESCRIPTION OF DRAWINGS

FIG. 1 shows the influence of orally administered anserine on the uric acid level in blood.

### DETAILED DESCRIPTION OF THE INVENTION

In the first aspect, the present invention provides a pharmaceutical composition for rapidly reducing the uric acid level in blood in a subject, which comprises anserine in an amount of greater than 50 mg to about 600 mg. The pharmaceutical composition of the invention can rapidly reduce the uric acid level in blood in a subject, thereby treating gout and/or ameliorating the symptoms associated with a high uric acid level in blood.

In the second aspect, the present invention provides a package, which comprises the pharmaceutical composition of the invention, and a label and/or a packing insert indicating that the composition is administered to a subject to provide anserine in an amount of greater than 50 mg to about 600 mg per dose. The package of the present invention is useful to rapidly reduce the uric acid level in blood in a subject, and thereby mating out and/or ameliorating the symptoms associated with a high uric acid level in blood.

In the third aspect, the present invention provides a method for rapidly reducing the uric acid level in blood in a subject, which comprises administering anserine in an amount of greater than 50 mg to about 600 mg to the subject. The method of the present invention can rapidly reduce the uric acid level in blood in a subject, thereby treating gout and/or ameliorating the symptoms associated with a high uric acid level in blood.

In the fourth aspect, the present invention provides a use of anserine in the manufacture of a medicament capable of rapidly reducing the uric acid level in blood in a subject, wherein the medicament comprises anserine in an amount of greater than 50 mg to about 600 mg. The medicament can rapidly reduce the uric acid level in blood in a subject, thereby treating gout and/or ameliorating the symptoms associated with a high uric acid level in blood.

Anserine may be naturally occurring or synthesized. In the present invention, anserine is administered to a subject in an amount of greater than 50 mg to about 600 mg, preferably from about 100 mg to about 500 mg, more preferably from about 150 mg to 250 mg, per dose, in order to achieve an efficacy of rapidly reducing the uric acid level in blood in the subject.

In the subject application, the term “rapidly reducing the uric acid level in blood” means that the reduced amount of the uric acid level in blood in a subject is about 0.5 to about 4 mg/dL, within one hour of the administration of anserine. The term “a subject” refers to a mammal, preferably a human. In addition, the term “symptoms related to a high level of uric acid” refers to, for example, muscle spasm, localized swelling, inflammation, joint pains, muscle fatigue, feelings of stress, myocardial infraction, or the combination thereof.

The pharmaceutical composition and medicament of the invention can be prepared by any conventional methods known in the art. In addition to the active ingredient, anserine, the pharmaceutical composition and medicament of the invention may comprise a pharmaceutically or physiologically acceptable excipient. Suitable pharmaceutically acceptable excipients include, but are not limited to, dextrin-maltose, lactose, sucrose, mannitol, starch, microcrystalline cellulose, carboxymethyl cellulose, talc, and magnesium stearate.

The pharmaceutical composition and medicament of the invention may be formulated in appropriate dosage forms, such as tablets, powder, granules, capsules, liquid, and suspension, for various administration routes. The pharmaceutical composition and medicament of the invention may be administered via any suitable routes, preferably via oral or parenteral route.

Any known packing and printing methods can be used to prepare the package of the invention.

### Examples

The invention will become apparent with reference to the following examples, which are purely exemplary, and should not be taken to limit the scope of the invention as described in the claims.

### Example 1

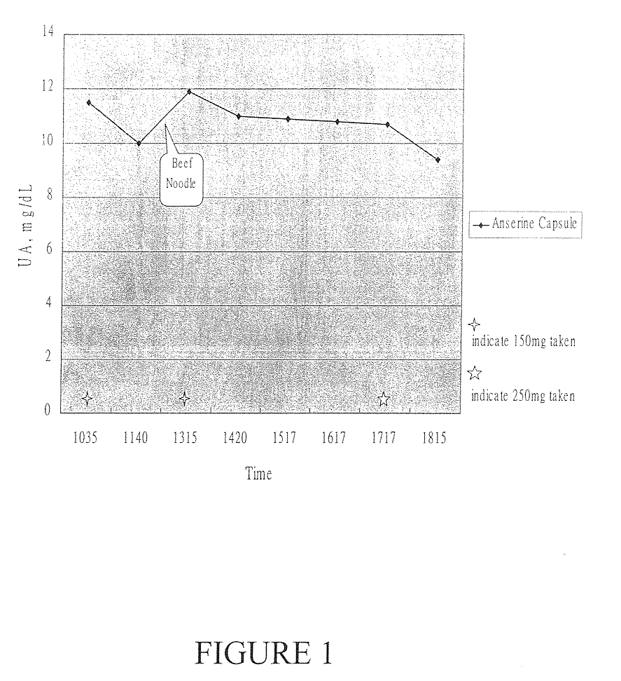

Anserine (GL Biochem Ltd., Shanghai, China) and dextrin-maltose were filled into a gelatin capsule (total weight 400 mg), and the capsule comprised 50 mg of anserine. A subject suffering from gout and having a high level of uric acid in blood took three capsules (a total 150 mg of anserine) two times a day and five capsules (a total 250 mg of anserine) once in the same day, so the total dosage of anserine taken by the subject daily was 550 mg. The uric acid level in blood was monitored by UroSpeed, a rapid sero-uric acid test strip (Apex Biotechnology Co., Inc. Hsinchu, Taiwan, ROC). The results are shown in Table 1, and further drawn in FIG. 1.

TABLE 1
Decrease in the uric acid level in blood one hour after anserine is
orally administered

| | | |
|---|---|---|
| | | Decrease in uric |
| | | acid level in |
| | | blood 1 hour after |
| | Uric acid level | anserine is orally |
| Anserine | in blood | administered |

| Time | dosage | (mg/dL) | (mg/dL) | (%) |
|---|---|---|---|---|
| 10:35 | 150 mg | 11.5 | 1.5 | 13.04% |
| 11:40 | — | 10.0 | | |
| 13:15 | 150 mg | 11.9 | 0.9 | 7.56% |
| 14:20 | — | 11.0 | | |
| 17:17 | 250 mg | 10.7 | 1.3 | 12.15% |
| 18:15 | — | 9.4 | | |

As shown in Table 1 and FIG. 1, the range of decrease in the level of uric acid in blood is from 0.9 mg/dL to 1.5 mg/dL one hour after the regimen of the capsule comprising anserine. One hour after the regimen of 150 mg of anserine at 10:35, the level of uric acid in blood decreased by 1.5 mg/dL. After the subject ate beef noodle at noon, the level of uric acid in the subject's blood increased to 11.9 mg/dL. One hour after regimen of 150 mg of anserine at 13:15, the level of uric acid in blood of the subject decreased by 0.9 mg/dL. One hour after consuming 250 mg of anserine at 17:17, the uric acid level in blood of the subject decreased by 1.3 mg/dL. The data given in Table 1 reveals that the administration of anserine can rapidly reduces the uric acid level in blood in a subject within one hour.

### Example 2

Fifteen voluntary adult subjects (nine males and six females 28 to 53 years old) having chronic hyperuricemia were tested in this oral administration test. These subjects stopped their regular medication for at least 3 days. The average uric acid level in blood of these subjects became greater than 9.3 mg/dl. These subjects were randomly separated in five groups, each comprising three subjects, to take anserine in the following dosages: 50 mg, 100 mg, 150 mg, 200 mg, and 250 mg, respectively. After one hour, the uric acid levels in the blood of the subjects were measured. It was found that the uric acid level in their blood decreased by 1.1 mg/dL to 1.3 mg/dL. Among the five groups, the decrease in the uric acid level in blood in the group taking 150 mg of anserine is the most significant one. The results are shown in Table 2.

TABLE 2
Influence on uric acid level in blood 1 hour after various dosages
of anserine are orally administered

| | | |
|---|---|---|
| | | Decrease in uric |
| | | acid in blood |
| | | 1 hour after |
| | Uric acid level in blood | anserine is orally |
| Anserine | (mg/dL) | administered |

| (mg/day) | 0 hr | 1 hr | (mg/dL) | (%) |
|---|---|---|---|---|
| 50 | 9.1 ± 1.3 | 7.9 ± 1.0 | 1.3 ± 0.5 | 13.7 |
| 100 | 8.8 ± 1.2 | 7.6 ± 1.4 | 1.2 ± 0.5 | 13.9 |
| 150 | 8.5 ± 0.7 | 7.3 ± 0.9 | 1.2 ± 0.2 | 14.6 |
| 200 | 10.1 ± 1.4 | 8.8 ± 1.4 | 1.3 ± 0.3 | 13.1 |
| 250 | 9.9 ± 0.9 | 8.8 ± 1.0 | 1.1 ± 0.3 | 11.5 |

What is claimed is:

1. A pharmaceutical composition for rapidly reducing the uric acid level in blood in a subject, which comprises anserine in an amount of greater than 50 mg to about 600 mg.

2. The pharmaceutical composition of claim 1, which comprises anserine in an amount of about 100 mg to about 400 mg.

3. The pharmaceutical composition of claim 2, which comprises anserine in an amount of about 150 mg to 250 mg.

4. The pharmaceutical composition of any one of claims 1 to 3, which is useful in treating gout and/or alleviating symptoms associated with a high uric acid level in blood.

5. The pharmaceutical composition of claim 4, wherein the symptom is selected from muscle spasm, localized swelling, inflammation, joint pains, muscle fatigue, feelings of stress, myocardial infraction, and the combination thereof.

6. A package comprising:
a pharmaceutical composition comprising anserine; and
a label and/or an insert indicating that the composition is administered to a subject to provide anserine in an amount of greater than 50 mg to about 600 mg per dose so as to treat gout and/or alleviate symptoms associated with a high uric acid level in blood.

7. The package according to claim 6, wherein the label and/or the insert indicate that the composition is administered to a subject to provide anserine in an amount of about 100 mg to about 500 mg.

8. The package according to claim 7, wherein the label and/or the insert indicate that the composition is administered to a subject to provide anserine in an amount of about 150 mg to about 250 mg.

9. The package according to claim 6, wherein the symptom is selected from muscle spasm, localized swelling, inflammation, joint pains, muscle fatigue, feelings of stress, myocardial infraction, and the combination thereof.

10. A method for rapidly reducing the uric acid level in blood in a subject, which comprises administering anserine in an amount of greater than 50 mg to about 600 mg to the subject.

11. The method according to claim 10, wherein anserine is administered in an amount of about 100 mg to about 400 mg.

12. The method according to claim 11, wherein anserine is administered in an amount of about 150 mg to about 250 mg.

13. The method according to claim 10, which is useful in treating gout and/or alleviating symptoms associated with a high uric acid level in blood.

14. The method according to claim 13, wherein the symptom is selected from muscle spasm, localized swelling, inflammation, joint pains, muscle fatigue, feelings of stress, myocardial infraction, and the combination thereof.
